(19) United States
(12) Patent Application Publication
(10) Pub. No.: US 2013/0295625 A1
(43) Pub. Date: Nov. 7, 2013

(54) Apparatus and Method for Treating Organic Waste

(71) Applicants: Chung-Hee Chang, Seoul (KR); Young-O Kim, Gyeonggi-Do (KR); Duk-Woo Jun, Seoul (KR)

(72) Inventors: Chung-Hee Chang, Seoul (KR); Young-O Kim, Gyeonggi-Do (KR); Duk-Woo Jun, Seoul (KR)

(73) Assignee: Hyundai Engineering & Construction Co., Ltd., Seoul (KR)

(21) Appl. No.: 13/879,879

(22) PCT Filed: Nov. 30, 2011
(86) PCT No.: PCT/KR2011/009211

§ 371 Date: Jul. 25, 2013

(30) Foreign Application Priority Data
Nov. 30, 2010 (KR) 10-2010-0120322

(51) Int. Cl. C12M 1/107

(52) U.S. Cl. CPC C12M 21/04
USPC 435/155; 435/290.4

(57) ABSTRACT

The present invention relates to an apparatus for treating organic waste including: a first circulation line having a first circulation pump connected thereon so as to supply a portion of the organic waste being acid fermented in an acid fermenter to a methane fermenter; a second circulation line having a second circulation pump connected thereon so as to supply a portion of anaerobic digestive fluid methane fermented in the methane fermenter to the acid fermenter; and vortex generating means having a plurality of first, second and third nozzles disposed in the methane fermenter so as to allow the anaerobic digestive fluid in the methane fermenter to be agitated by vortices generated thereof.

### BACKGROUND OF THE INVENTION

1. Field of the Invention

The present invention relates to an apparatus and method for treating organic waste, and more particularly, to an apparatus and method for treating organic waste wherein the organic waste is agitated by means of the formation of vortices in an anaerobic reactor and digestive fluid in a methane fermenter is introduced into a buffer tank in such a manner as to be passed through a membrane device in a two-way alternating fashion every given period of time, such that through the repeated circulation of the digestive fluid along the buffer tank and the membrane device, the digestive fluid becomes high concentrated and the final high concentrated liquid is supplied to the anaerobic reactor.

2. Background of the Related Art

Generally, the growth of earnings, the changes in the consumption trends and the development of the distribution structure are accompanied by industrial development, and thus, municipal waste becomes drastically increased.

So as to treat the municipal waste, accordingly, there have been proposed various methods for landfilling the municipal waste on landfills, incinerating the municipal waste, and re-processing and recycling the municipal waste.

The landfilling method has some problems that after the treatment, geological pollution, harmful gas generation, water pollution and the like are happened and the economical load for occupying the landfills is increased.

The incineration method has some problems that the costs consumed for the incineration are increased, air pollution is caused upon the incineration, and the kinds of waste to be treated are limited.

The recycling method looks like the best treatment method, but is selectively adopted in accordance with the recycling efficiencies. That is, it is determined whether the recycling process is performed or not in accordance with the economical efficiencies of the recycling treatment costs, secondary environmental pollution problems occurring during the recycling treatment, and the degrees of satisfaction in the qualities of final recycling products.

In the past, high concentrated organic waste (for example, food waste, sewage sludge, waste water sludge, livestock waste and the like) produced from normally food waste is landfilled. However, the landfilling of the high concentrated organic waste causes water pollution and odor generation by decay, which becomes one of serious environmental pollution causes. After the landfilling, further, non-economical, non-sanitary, and non-environmental problems are happened such as the changes in the surrounding soil qualities, the generation of odor by the decay of the organic matters, and air and water pollution. In recent days, accordingly, the method for landfilling the high concentrated organic waste has been prohibited in many countries.

As greenhouse and energy problems have been recently emerged, furthermore, methods for producing bio-energy using high concentrated organic waste have been introduced and utilized all over the world. That is, the high concentrated organic waste is not considered as an object to be simply treated, but considered as an energy source capable of producing renewable energy. Thus, a new method has been suggested wherein methane gas is produced from the organic waste through an anaerobic digestion process, and digestive fluid is purified and discharged, and so as to perform the method, accordingly, an apparatus for treating the organic waste has been developed.

Therefore, conventional apparatuses for treating organic waste have been proposed wherein the methane gas is extracted from the organic waste, thereby removing other toxic gas and sludge, and the digestive fluid is purified and discharged for recycling.

Referring simply to one of the conventional apparatuses for treating organic waste, the organic waste is reacted with microorganisms in an anaerobic reactor composed of an acid fermenter and a methane fermenter, and through the reaction, methane gas is generated and stored separately. The digestive fluid discharged from the methane fermenter is utilized as compost or purified through a biological or physicochemical process.

So as to improve the anaerobic digestion efficiency and simplify the digestive fluid treatment, recently, a membrane-combined anaerobic digestion technology has been introduced and suggested wherein a solid/liquid separation membrane is combined with an anaerobic digestion tank. According to the membrane-combined anaerobic digestion technology, the digestive fluid discharged from the methane fermenter is separated into solid and liquid states in the membrane device and at the same time, the separated solid is returned to the anaerobic digestion tank.

In this case, the acid fermenter and the methane fermenter have respective agitators having agitating wings and an agitating pump so as to improve the reaction of the organic waste with the microorganisms.

Also, the concentrated liquid filtered through the membrane device is returned directly to the methane fermenter and the acid fermenter so as to maintain the concentrations of the microorganisms therein and increase the contact time between the biodegradable solid having slow decomposition speed and the microorganisms, thereby enhancing the treatment efficiency.

Further, the digestive fluid is introduced into the membrane device only in one direction and is separated therein into solid and liquid.

According to the conventional apparatus for treating organic waste under the above-mentioned configuration, the agitation through the agitators is actively performed only at the positions where the agitating wings are disposed, but the agitation efficiency is remarkably lowered in the positions distant from the agitating wings. Further, the concentrated liquid concentrated one time in the membrane device is returned to the anaerobic reactor, but after a given period of time, it is observed that the fermentation and decomposition efficiencies in the anaerobic reactor are decreased drastically, thereby undesirably causing the temperature loss of the methane fermented liquid, the impact load caused by the abrupt concentrating operation, and large amounts in circulation for filtering. Moreover, since the digestive fluid is passed through the membrane device in one direction, pollutants become increasingly collected to decrease a flow rate supplied therein and the exchange period of the membrane device becomes also shortened.

### SUMMARY OF THE INVENTION

Accordingly, the present invention has been made in view of the above-mentioned problems occurring in the prior art, and it is an object of the present invention to provide an apparatus and method for treating organic waste wherein the organic waste is agitated by means of agitators as well as the formation of vortices, thereby improving the agitating performance for the organic waste and optimizing the reaction of the organic waste with microorganisms.

It is another object of the present invention to provide an apparatus and method for treating organic waste wherein as digestive fluid or low concentrated liquid is repeatedly circulated through a membrane device and a buffer tank, it becomes high concentrated and is then supplied to an anaerobic reactor, and wherein the internal negative pressure in the buffer tank is compensated to permit the digestive fluid or the concentrated liquid to be gently discharged.

It is still another object of the present invention to provide an apparatus and method for treating organic waste wherein the advancing direction of the digestive fluid or the concentrated liquid introduced into the membrane device is changed every given period of time in a two-way alternating fashion so as to remarkably reduce the collection of the pollutants in the membrane device, thereby constantly maintaining the efficiency of the membrane device and also extending the life cycle of the membrane device.

To accomplish the above objects, according to an aspect of the present invention, there is provided an apparatus for treating organic waste, in which an acid fermenter, a methane fermenter, a buffer tank and a membrane device are disposed, the apparatus including: a first circulation line having a first circulation pump connected thereon so as to supply a portion of the organic waste being acid fermented in the acid fermenter to the methane fermenter; a second circulation line having a second circulation pump connected thereon so as to supply a portion of anaerobic digestive fluid methane fermented in the methane fermenter to the acid fermenter; and vortex generating means having a plurality of first, second and third nozzles disposed in the methane fermenter so as to allow the anaerobic digestive fluid in the methane fermenter to be agitated by vortices generated thereof, wherein the vortex generating means comprises: a first nozzle part having the plurality of first nozzles disposed at a given angle on a first nozzle pipe so as to form water flows in one direction in the anaerobic digestive fluid fermented in the methane fermenter; a second nozzle part having the plurality of second nozzles disposed at a given angle on a second nozzle pipe so as to form water flows in the opposite direction to the injection direction of the first nozzle part; and a first vortex circulation line adapted to supply the anaerobic digestive fluid on the upper portion of the methane fermenter to the first and second nozzle pipes, whereby the water flows are formed alternatively in the one direction and opposite direction to the one direction to generate vortices in the methane fermenter.

According to the present invention, preferably, the apparatus further includes: a sorting crusher adapted to sort and crush the organic waste; a mill adapted to grind the crushed organic waste through the sorting crusher to appropriate sizes capable of being introduced into the acid fermenter; a screen adapted to filter the organic waste of more than a predetermined size from the organic waste passed through the mill; a storage tank adapted to temporarily store the organic waste passed through the screen; heaters disposed in the acid fermenter and the methane fermenter so as to raise the organic waste in the acid fermenter and the methane fermenter up to a given temperature; a gas storage tank adapted to store the methane gas generated from the methane fermenter therein; and a boiler adapted to receive the methane gas stored in the gas storage tank so as to supply heat to the heaters.

According to the present invention, preferably, the first vortex circulation line includes: a first vortex circulation pump connected thereon to supply the anaerobic digestive fluid on the upper portion of the methane fermenter to the first and second nozzle pipes; and a pair of engagement valves adapted to open the first and second nozzle pipes alternatively.

According to the present invention, preferably, the first and second nozzle pipes are disposed on the bottom portion of the methane fermenter in such a manner as to be bent at a given curvature toward the center of the methane fermenter.

According to the present invention, preferably, the plurality of first nozzles and second nozzles have an upward injection angle (θ) in a range of 30° to 45°.

According to the present invention, preferably, the vortex generating means further includes: a third nozzle part having the plurality of third nozzles disposed at a given angle on a third nozzle pipe located on the upper portion of the methane fermenter; and a second vortex circulation line adapted to supply the anaerobic digestive fluid on the lower portion of the methane fermenter to the third nozzle pipe.

According to the present invention, preferably, the second vortex circulation line has a second vortex circulation pump connected thereon so as to supply the anaerobic digestive fluid on the lower portion of the methane fermenter to the third nozzle pipe.

According to the present invention, preferably, the plurality of third nozzles have a given upward and vertical or oblique injection angle.

According to the present invention, preferably, the plurality of third nozzles have an upward or downward injection angle (θ) in a range of 30° to 45°.

According to the present invention, preferably, the vortex generating means further includes a controller adapted to control the first vortex circulation pump and the second vortex circulation pump in such a manner as to be operated alternatively and to control the engagement valves in such a manner as to operate the first nozzle part and the second nozzle part alternatively.

According to the present invention, preferably, the vortex generating means further includes a plurality of baffles disposed along the side surface of the lower portion of the methane fermenter so as to provide interferences to the flows of the vortices.

According to the present invention, preferably, the methane fermenter includes: a plurality of sludge grooves formed along the edge portion of the bottom surface thereof so as to collect the sludge falling down by the contact with the baffles; and sludge discharging lines connected to the sludge grooves so as to discharge sludge therethrough.

According to the present invention, preferably, the bottom surface of the methane fermenter is inclined downwardly from the center to the edge side thereof.

According to the present invention, preferably, the methane fermenter further includes a second agitator having second agitating wings disposed therein and a second agitating motor for rotating the second agitating wings.

According to the present invention, preferably, the acid fermenter includes a first agitator having first agitating wings disposed therein and a first agitating motor for rotating the first agitating wings.

According to the present invention, preferably, the acid fermenter further includes an agitation circulation line adapted to connect the upper and lower portions thereof and having an agitation circulation pump connected thereon so as to supply the organic waste from the upper portion thereof to the lower portion thereof.

According to the present invention, preferably, the first circulation line is connected to the first vortex circulation line from the acid fermenter.

According to the present invention, preferably, the connected point between the first circulation line and the first vortex circulation line is disposed before the engagement valves.

According to the present invention, preferably, the second circulation line is connected to the agitation circulation line from the methane fermenter.

According to the present invention, preferably, the buffer tank includes a gas holder disposed on the upper portion thereof so as to compensate for a negative pressure generated when the digestive fluid stored therein is discharged.

According to the present invention, preferably, the gas holder is formed of a soft material, and if the negative pressure is generated inside the buffer tank, the gas holder is contracted through the discharging of the gas collected therein.

According to the present invention, preferably, the apparatus further including injection direction change means adapted to reversely change the injection direction of the digestive fluid supplied to the membrane device every given period of time, wherein the injection direction change means includes: a second digestive fluid introduction line branched from a first digestive fluid introduction line connected to the buffer tank in such a manner as to be connected to the other end of the membrane device; a second concentrated liquid return line connected to the membrane device so as to connect a first concentrated liquid return line disposed to supply concentrated liquid to the acid fermenter and the methane fermenter thereto; a first injection valve mounted on the first digestive fluid introduction line; a first discharge valve mounted on the first concentrated liquid return line; a second injection valve mounted on the second digestive fluid introduction line; a second discharge valve mounted on the second concentrated liquid return line; and a controller adapted to control a supply pump mounted on the first digestive fluid introduction line, the first and second injection valves, and the first and second discharge valves.

According to the present invention, preferably, the first concentrated liquid return line is connected to a supply line so as to circulate the concentrated liquid to the buffer tank.

According to the present invention, preferably, the apparatus further includes: a circulation valve mounted on the first concentrated liquid return line so as to introduce the concentrated liquid conveyed through the first concentrated liquid return line into the supply line or to stop the introduction into the supply line; a first supply valve mounted on the first concentrated liquid return line so as to introduce the concentrated liquid conveyed through the first concentrated liquid return line into the acid fermenter or to stop the introduction into the acid fermenter; a second supply valve mounted on the first concentrated liquid return line so as to introduce the concentrated liquid conveyed through the first concentrated liquid return line into the methane fermenter or to stop the introduction into the methane fermenter; and a shut-off valve adapted to be open and closed so as to stop the movement of the digestive fluid discharged from the methane fermenter.

According to the present invention, preferably, if the circulation valve is open, the shut-off valve, the first supply valve and the second supply valve become closed, and if the circulation valve is closed, the first supply valve and the second supply valve become open.

According to the present invention, preferably, the shut-off valve is open only when the digestive fluid is supplied to the buffer tank.

According to the present invention, preferably, the membrane device includes: a frame; one or more membrane filtration modules disposed on the frame and each having a casing having a discharge hole formed to discharge filtered water therethrough and a plurality of tubular type filtration membranes mounted inside the casing; a first header disposed on one ends of the membrane filtration modules and connected to the first digestive fluid introduction line; and a second header disposed on the other ends of the membrane filtration modules and connected to the first concentrated liquid return line.

According to the present invention, preferably, the membrane device further includes: a first flow meter mounted on the first digestive fluid introduction line so as to measure an injection flow rate of the digestive fluid; and a second flow meter mounted on the first concentrated liquid return line so as to measure a discharge flow rate of the concentrated liquid.

According to the present invention, preferably, the membrane device further includes: a first manometer mounted on the first digestive fluid introduction line so as to measure an injection pressure of the digestive fluid; and a second manometer mounted on the first concentrated liquid return line so as to measure a discharge pressure of the digestive fluid.

According to the present invention, preferably, the membrane device further includes third flow meters adapted to measure a discharge flow rate of the filtered water discharged from the membrane filtration modules.

According to the present invention, preferably, the membrane device further includes washing means adapted to backwash the membrane device in such a manner as to be activated when a flow rate difference between the flow rate values measured through the first and second flow meters is calculated by the controller and is smaller than a given reference value.

According to the present invention, preferably, the washing means includes: a wash water supply line connected to the second digestive fluid introduction line; a water tank connected to the wash water supply line; and a backwash pump adapted to pressurize and convey the wash water stored in the water tank to the wash water supply line.

According to the present invention, preferably, the washing means further includes at least one of: a discharge tank adapted to temporarily store the filtered water discharged from the membrane device; a chemical tank adapted to add washing chemicals to the wash water supply line; and an air compressor adapted to supply compressed air to the wash water supply line.

According to the present invention, preferably, the washing means further includes: a fifth valve mounted on the first concentrated liquid return line in such a manner as to be closed during the backwashing; and a first wash fluid return line branched from the first concentrated liquid return line so as to introduce the wash water thereinto when the fifth valve is closed in such a manner as to be connected to a raw water storage tank and the chemical tank.

To accomplish the above objects, according to another aspect of the present invention, there is provided a method for treating organic waste including: a first step wherein the organic waste is supplied and fermented to an acid fermenter and a methane fermenter; a second step wherein the methane gas generated in the first step is stored in a gas storage tank; a third step wherein the digestive fluid discharged from the methane fermenter is introduced and discharged into/from a buffer tank through a supply line; a fourth step wherein the digestive fluid stored in the buffer tank is injected into the membrane device by means of a supply pump and is separated into the concentrated liquid and filtered water and wherein the injection direction of the digestive fluid is changed reversely every given period of time; and a sixth step wherein the separated concentrated liquid is returned to the acid fermenter and the methane fermenter.

According to the present invention, preferably, in the first step vortices are formed in a clockwise or counterclockwise direction by means of a plurality of first and second nozzles, and vortices are formed in different directions by means of a plurality of third nozzles.

According to the present invention, preferably, in the first step a portion of the digestive fluid of the methane fermenter is circulated to the acid fermenter through a second circulation line connecting the acid fermenter and the methane fermenter, thereby being maintained in a range of pH4.0 to pH4.5.

According to the present invention, preferably, in the third step the negative pressure generated when the digestive fluid is discharged from the buffer tank is compensated by the contraction of a gas holder through the gas discharging therefrom, the gas holder being disposed inside the buffer tank in such a manner as to be expanded by collecting the gas generated in the buffer tank.

According to the present invention, preferably, the fourth step further includes the steps of: measuring a flow rate difference or a pressure difference between both ends of tubular type filtration membranes disposed in the membrane device; comparing the measured difference with a given reference value; after the comparing result, if the flow rate different or the pressure difference is smaller than the given reference value, stopping the fourth and sixth steps; and backwashing the tubular type filtration membranes for a given period of time.

According to the present invention, preferably, the backwashing step includes: a water washing step of pressurizing and injecting wash water stored in a water tank into the tubular type filtration membranes by means of the activation of a backwash pump so as to perform water washing; and a chemical washing step of adding chemicals to the wash water so as to perform chemical washing if a washing period in the water washing step is smaller than a given reference value.

According to the present invention, preferably, the backwashing step further includes the steps of: returning the wash water after the water washing to a raw water storage tank; and returning the wash water after the chemical washing to a chemical tank.

According to the present invention, preferably, the method further includes a fifth step wherein it is determined whether the concentrated liquid discharged from the membrane device is repeatedly filtered.

According to the present invention, preferably, in the fifth step if the concentrated liquid is not repeatedly filtered, it is returned to the third step wherein the methane fermented liquid is introduced into the buffer tank through the supply line.

According to the present invention, preferably, in the fifth step if the concentrated liquid is repeatedly filtered, it is moved to the sixth step.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a schematic view showing a configuration of an apparatus for treating organic waste according to the present invention;

FIG. 2 is a schematic view showing an acid fermenter and a methane fermenter in FIG. 1;

FIGS. 3 and 4 are front and plane views showing the methane fermenter in FIG. 2;

FIGS. 5 to 7 are schematic views showing vortices formed inside the acid fermenter and the methane fermenter in FIG. 2;

FIGS. 8 and 9 are schematic side sectional views showing the operational states of a gas holder disposed inside a buffer tank in FIG. 1;

FIG. 10 is a schematic sectional view showing a membrane filtration module according to the present invention; and

FIG. 11 is a flowchart showing a method for treating organic waste using the apparatus for treating organic waste of FIG. 1.

### DETAILED DESCRIPTION OF THE PREFERRED EMBODIMENT

Hereinafter, an explanation on an apparatus and method for treating organic waste according to the present invention will be in detail given with reference to the attached drawing.

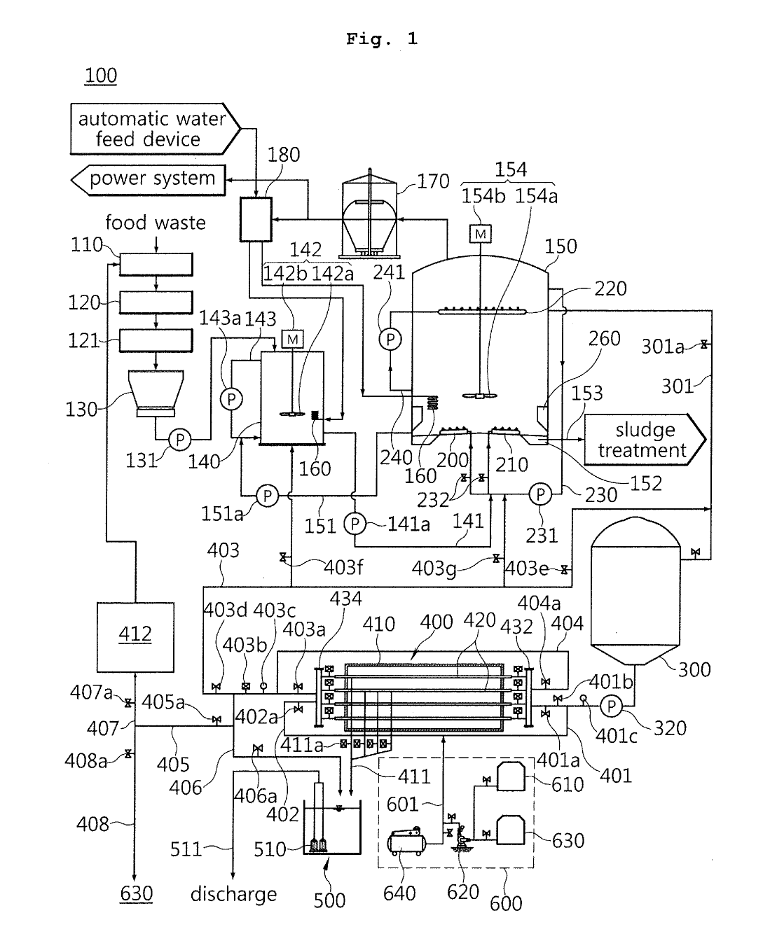

First, as shown in FIG. 1, an apparatus 100 for treating organic waste according to the present invention largely includes a sorting crusher 110, a mill 120, a screen 121, storage tank 130, an acid fermenter 140, a methane fermenter 150, heaters 160 disposed inside the acid fermenter 140 and the methane fermenter 150, a gas storage tank 170, a boiler 180, a buffer tank 300, a supply pump 320, a membrane device 400, a first digestive fluid introduction line 401, injection direction change means, a first concentrated liquid return line 403, a discharge tank 500 and washing means 600.

In this case, the sorting crusher 110 serves to sort foreign matters not biologically degraded such as synthetic resin, metals and the like contained in the organic waste and to crush and break large solid matters in the organic waste.

The mill 1120 serves to finely grind the crushed organic waste, and since the organic waste grinded through the mill 120 contains water of 80% to 85% therein, the organic waste has relatively good fluidity.

The screen 121 serves to filter the vinyl and solid matters and inert materials of more than 5 mm therethrough, thereby providing better fluidity.

The storage tank 130 serves to temporarily store the organic waste discharged from the mill 120 therein and has a pump 131 adapted to convey the organic waste therein to the acid fermenter 140.

The heaters 160 are connected to the boiler 180 to heat the organic waste in the acid fermenter 140 up to a temperature in a range of approximately 60° C. to 100° C. and to heat the organic waste in the methane fermenter 150 up to a temperature in a range of approximately 50° C. to 60° C., which allows the organic waste in which oil is contained in large quantities to be fermented to an optimal degree.

The gas storage tank 170 serves to store the methane gas generated from the acid fermenter 140 and the methane fermenter 150 therein. The methane gas stored in the gas storage tank 170 is partially supplied to the boiler 180 and is partially supplied to a high purity gas refinery or a power system for production of power.

The boiler 180 serves to supply heat to the heaters 160 with the energy sources of the methane gas stored in the gas storage tank 170.

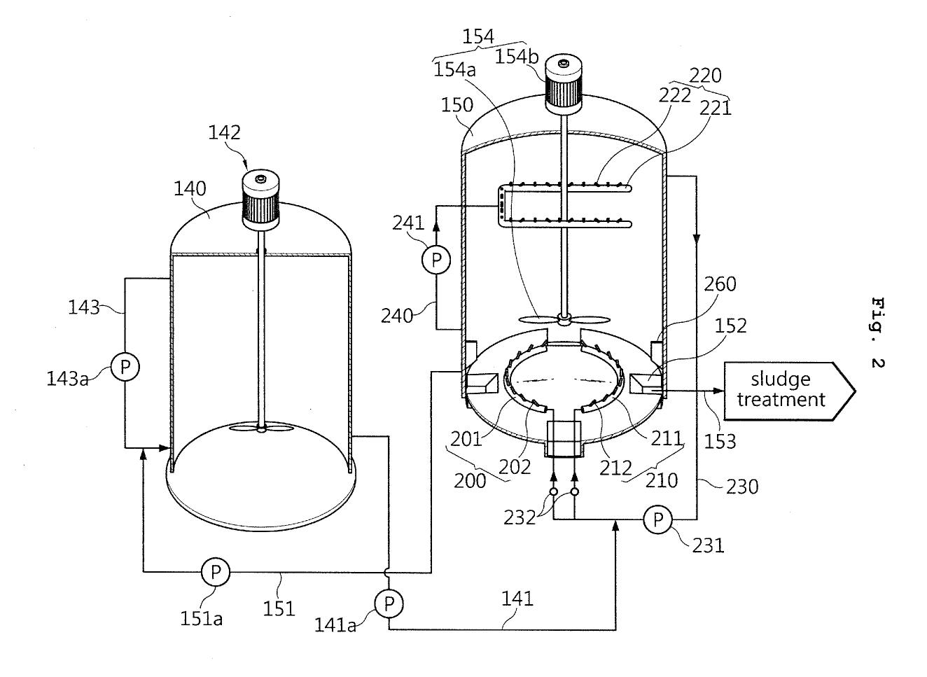

On the other hand, as shown in FIG. 2, the acid fermenter 140 includes a first circulation line 141 adapted to supply the acid fermented organic matters to the methane fermenter 150, a first agitator 142 disposed at the inside thereof, and an agitation circulation line 143 adapted to circulate the organic waste disposed in the upper portion thereof to the lower portion thereof.

In this case, the first agitator 142 includes first agitating wings 142a placed inside the acid fermenter 140 and a first agitating motor 142b for rotating the first agitating wings 142a. At this time, the first agitating motor 142b desirably has a range between 15 rpm and 25 rpm. In more detail, if the first agitating motor 142b has the range of more than 25 rpm, the internal pressure thereof is raised to cause a dangerous state, while if the first agitating motor 142b has the range of less than 15 rpm, the agitation performance thereof is deteriorated.

Further, the agitation circulation line 143 is connected from the upper portion of the acid fermenter 140 to the lower portion thereof and is connected to an agitation circulation pump 143a adapted to circulate the organic waste disposed in the upper portion of the acid fermenter 140 to the lower portion thereof.

The first circulation line 141 is connected to a first vortex circulation line 230 of the methane fermenter 150 as will be discussed later and is connected to a first circulation pump 141a adapted to supply the acid fermented organic matters in the acid fermenter 140 to the methane fermenter 150.

In this case, the first circulation line 141 desirably circulates the acid fermented organic matters from the lower portion of the acid fermenter 140 to the lower portion of the methane fermenter 150, and at this time, the circulation is carried out every approximately 2 hours to 3 hours.

On the other hand, as shown in FIGS. 2 to 4, the methane fermenter 150 includes: vortex generating means by which anaerobic digestive fluid is mixed and fermented with vortices; a second agitator 154 disposed at the inside thereof; and a second circulation line 151 adapted to circulate a portion of the anaerobic digestive fluid fermented therein to the acid fermenter 140.

In this case, the second agitator 154 includes second agitating wings 154a placed inside the methane fermenter 150 and a second agitating motor 154b for rotating the second agitating wings 154a. At this time, the second agitating motor 154b desirably has a range between 15 rpm and 25 rpm. In more detail, if the second agitating motor 154b has the range of more than 25 rpm, the internal pressure thereof is raised to cause a dangerous state, and contrarily, if the second agitating motor 154b has the range of less than 15 rpm, the agitation performance thereof is deteriorated.

The vortex generating means includes first and second nozzle parts 200 and 210 disposed on the bottom portion of the methane fermenter 150, a third nozzle part 220 disposed on the upper portion of the methane fermenter 150 so as to perform the agitation in the methane fermenter 150, a first vortex circulation line 230 adapted to supply the anaerobic digestive fluid in the upper portion of the methane fermenter 150 to the first and second nozzle parts 200 and 210, and a second vortex circulation line 240 adapted to supply the anaerobic digestive fluid in the lower portion of the methane fermenter 150 to the third nozzle part 220.

In this case, the first vortex circulation line 230 is connected from the upper portion of the methane fermenter 150 to the first and second nozzle parts 200 and 210 in the lower portion of the methane fermenter 150 and has a first vortex circulation pump 231 connected to supply the anaerobic digestive fluid in the upper portion of the methane fermenter 150 to the first and second nozzle parts 200 and 210. Further, a pair of engagement valves 232 is disposed to open the first and second nozzle parts 200 and 210 alternatively.

In this case, desirably, the first vortex circulation pump 231 has a capacity capable of circulating a higher flow rate by 6 times to 8 times than an amount of organic waste introduced from the acid fermenter 140 so as to prevent the anaerobic microorganism flocs from being broken.

Also, the first nozzle part 200 includes a first nozzle pipe 201 connected to the first vortex circulation line 230 and a plurality of first nozzles 202 disposed on the first nozzle pipe 201 slantly to an angle of 30° to 45° so as to form water flows in an upward and clockwise direction in the drawings.

Also, the second nozzle part 210 includes a second nozzle pipe 211 connected to the first vortex circulation line 230 and a plurality of second nozzles 212 disposed on the second nozzle pipe 211 slantly to an angle of 30° to 45° so as to form water flows in an upward and counter-clockwise direction in the drawings.

The engagement valves 232 are controlled by means of a controller (not shown) as will be discussed later.

The second vortex circulation line 240 is connected from the lower portion of the methane fermenter 150 to the third nozzle part 220 in the upper portion of the methane fermenter 150 and has a second vortex circulation pump 241 connected to supply the organic matters in the lower portion of the methane fermenter 150 to the third nozzle part 220 when the first vortex circulation line 230 is not operated well in a state of emergency. At this time, the second vortex circulation line 240 desirably supplies the anaerobic digestive fluid from a point of ⅓ in height from the lower end of the methane fermenter 150 to a point of ⅔. Further, the second vortex circulation pump 241 has a capacity capable of circulating a higher flow rate by 2 times to 4 times than an amount of organic waste introduced from the acid fermenter 140.

The third nozzle part 220 is prepared when the first and second nozzle parts 200 and 210 stop by malfunctions and thus serves to keep the agitating operation in a state of emergency where the first and second nozzle parts 200 and 210 stop. Of course, even when the first and second nozzle parts 200 and 210 are normally operated, the third nozzle part 220 can be operated for improving the agitating performance. Also, the second vortex circulation line 240 is operated cooperatively with the third nozzle part 220.

The third nozzle part 220 includes a third nozzle pipe 221 connected to the second vortex circulation line 240 and a plurality of third nozzles 222 disposed on the third nozzle pipe 221.

In this case, the third nozzle pipe 221 is branched into two pipes inside the methane fermenter 150, and the branched pipes are bent as shown in the drawing. For example, the branched pipes may be formed like the curvatures of the first and second nozzle pipes 201 and 211.

Further, the third nozzles 222 are disposed upwardly on the third nozzle pipe 221 in such a manner as to have a perpendicular or oblique injection angle.

In another example, the third nozzles 222 may be disposed upwardly or downwardly on the branched third nozzle pipe 221 in such a manner as to have an injection angle of 30° to 45°.

The second circulation line 151 is connected from the methane fermenter 150 to the agitation circulation line 143 of the acid fermenter 140 and has a second circulation pump 151a connected thereon so as to circulate a portion of the organic matters in the methane fermenter 150 to the acid fermenter 140.

In this case, desirably, the second circulation line 151 circulates the anaerobic digestive fluid from the lower portion of the methane fermenter 150 to the lower portion of the acid fermenter 140.

The second circulation line 151 circulates a portion of the anaerobic digestive fluid from the methane fermenter 150 to the acid fermenter 140. Since the acid fermented organic matters agitated in the acid fermenter 140 have the properties of strong acids in a range of pH3 to pH4, the fermentation in the acid fermenter 140 may be not carried out gently, and as a portion of the anaerobic digestive fluid from the methane fermenter 150 is circulated to the acid fermenter 140, the acid degree in the acid fermenter 140 is lowered.

On the other hand, the operations of the first and second vortex circulation pumps 231 and 241 and the engagement valves 232 in the vortex generating means are controlled by means of the controller (not shown). Of course, the controller may be disposed to control all motors, pumps and valves mounted on the apparatus 100 for treating organic waste.

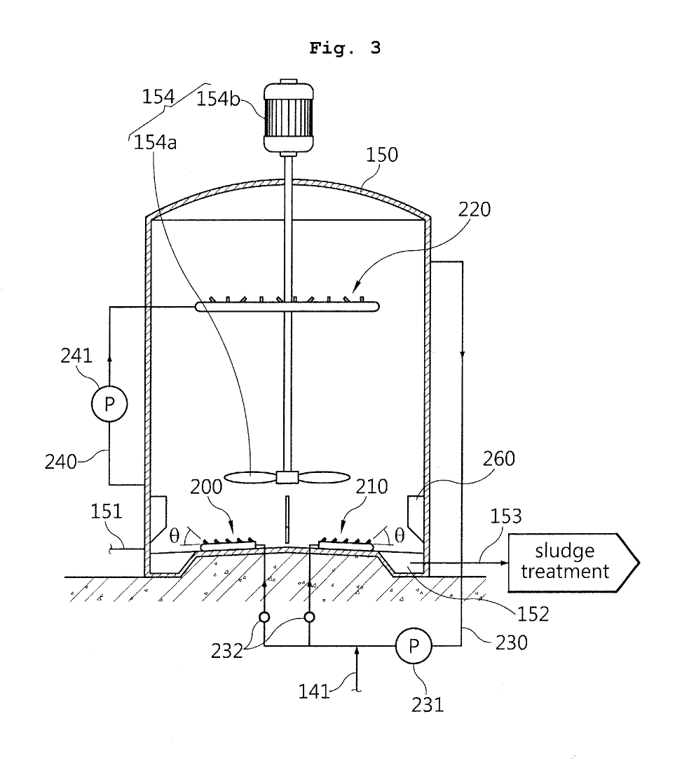

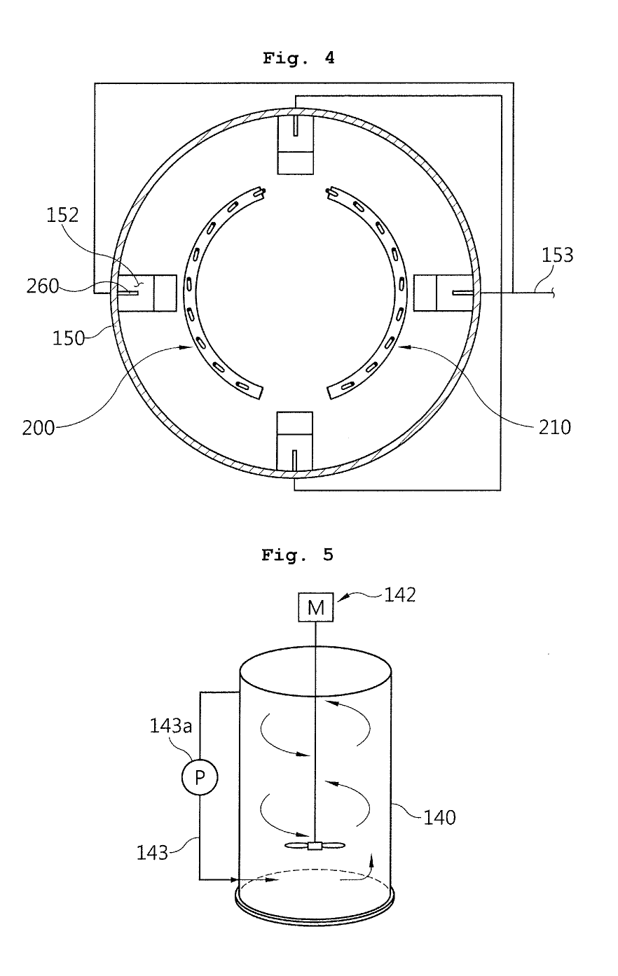

Also, as shown in FIGS. 3 and 4, the vortex generating means further includes a plurality of baffles 260 disposed along the side surface of the lower portion of the methane fermenter 150 so as to provide interferences to the flows of the vortices formed by means of the plurality of nozzles 202, 212, 222.

In this case, the methane fermenter 150 includes a plurality of sludge grooves 152 formed along the edge portion of the bottom surface thereof and sludge discharging lines 153 connected to the sludge grooves 152 so as to discharge sludge therethrough, and the bottom surface of the methane fermenter 150 is inclined downwardly from the center to the edge side thereof.

The sludge grooves 152 are desirably formed below the baffles 260, such that the sludge falling down by the baffles 260 can be collected directly on the sludge grooves 152.

Moreover, the downwardly inclined bottom surface of the methane fermenter 150 allows the sludge flowing on the bottom surface thereof to be moved to the edge side of the bottom surface thereof and to be thus collected on the sludge grooves 152.

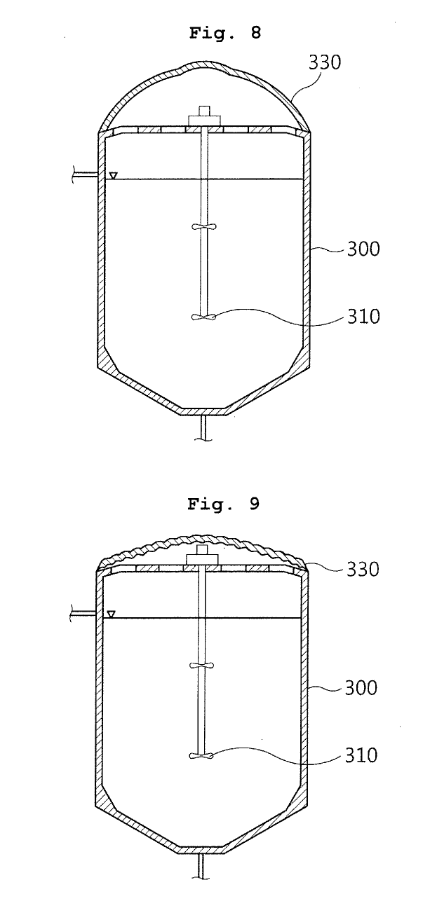

On the other hand, the buffer tank 300 is disposed to receive the digestive fluid discharged from the methane fermenter 150 through a supply line 301 and has a third agitator 310 adapted to agitate the digestive fluid so as to prevent the digestive fluid from being settled. Further, as shown in FIGS. 8 and 9, the buffer tank 300 includes a gas holder 330 disposed at the inside thereof and adapted to collect and expand the methane gas generated from the buffer tank 300 so as to compensate for the negative pressure generated when the digestive fluid is forcedly discharged to the membrane device 400 by means of a supply pump 320. In this case, the supply line 301 is connected to a first concentrated liquid return line 403 as will be discussed later. Also, the functions of the buffer tank 300 for generating the high concentrated liquid will be explained upon the description of the membrane device 400.

The gas holder 330 is formed of a soft material capable of expanding and contracting and is mounted on the upper portion of the buffer tank 300. If the negative pressure is generated inside the buffer tank 300, the gas holder 330 collects and expands the methane gas so as to compensate for the negative pressure generated. Of course, if the digestive fluid is introduced into the buffer tank 300 to form a positive pressure, the gas holder 330 becomes contracted, while discharging the methane gas contained therein therefrom. As another example of the gas holder 330, a panel may be slidingly disposed to close a portion of the space inside the buffer tank 300 and so as to compensate for the negative pressure, the methane gas is collected in the closed space to make the panel slide downwardly.

Meanwhile, the first digestive fluid introduction line 401 connects the buffer tank 300 and the membrane device 400 and serves as a moving passage of the digestive fluid. Further, the first digestive fluid introduction line 401 has a first flow meter 401b mounted thereon so as to measure an injection flow rate of the digestive fluid. Also, the first digestive fluid introduction line 401 has a first manometer 401c mounted thereon so as to measure an injection pressure of the digestive fluid.

The supply pump 320 is mounted on the first digestive fluid introduction line 401 and serves to pressurizedly convey the digestive fluid stored in the buffer tank 300 to the membrane device 400. The supply pump 320 is inverter controlled by means of the controller (not shown) in such a manner as to be connected to the buffer tank 300 and the first, second and third flow meters 401b, 403b, and 411a or the first and second manometers 401c and 403 and to be thus adjustable at the pumping speed.

The membrane device 400 serves to separate the digestive fluid conveyed through the supply pump 320 into concentrated liquid and filtered water. According to the present invention, as shown in FIG. 1, the membrane device 400 includes a frame 410, membrane filtration modules 420, and first and second headers 432 and 434.

In this case, the frame 410 serves to retain and support the membrane filtration modules 420.

Each of the first and second headers 432 and 434 is formed of a cylindrical body having given internal space therein. The first header 432 is adapted to allow the first digestive fluid introduction line 401 to communicate with the membrane filtration modules 420, and the second header 434 is adapted to allow the membrane filtration modules 420 to communicate with the first concentrated liquid return line 403. Accordingly, the digestive fluid introduced into the first header 432 through the first digestive fluid introduction line 401 is distributed to the membrane filtration modules 420, and the digestive fluid discharged from the membrane filtration modules 420 is collected to the second header 434 and is then discharged to the first concentrated liquid return line 403.

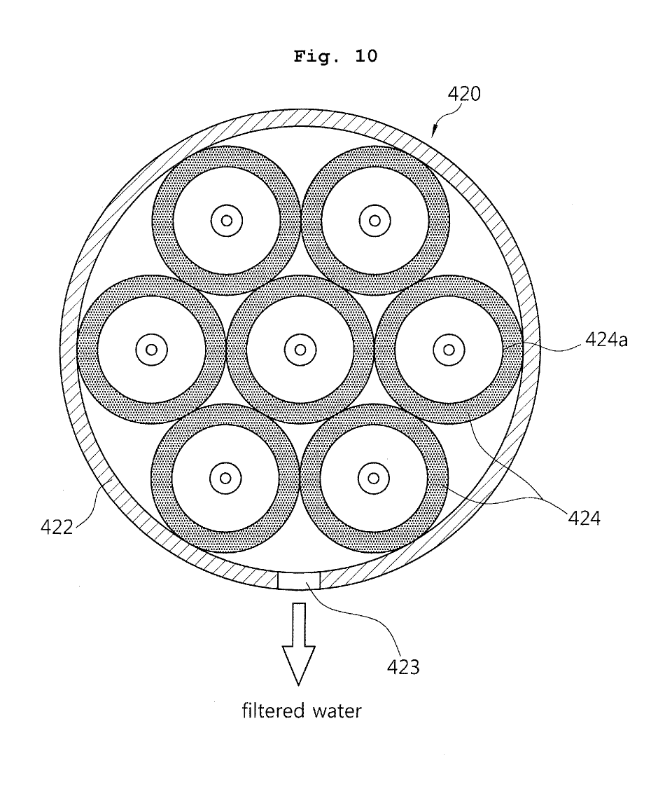

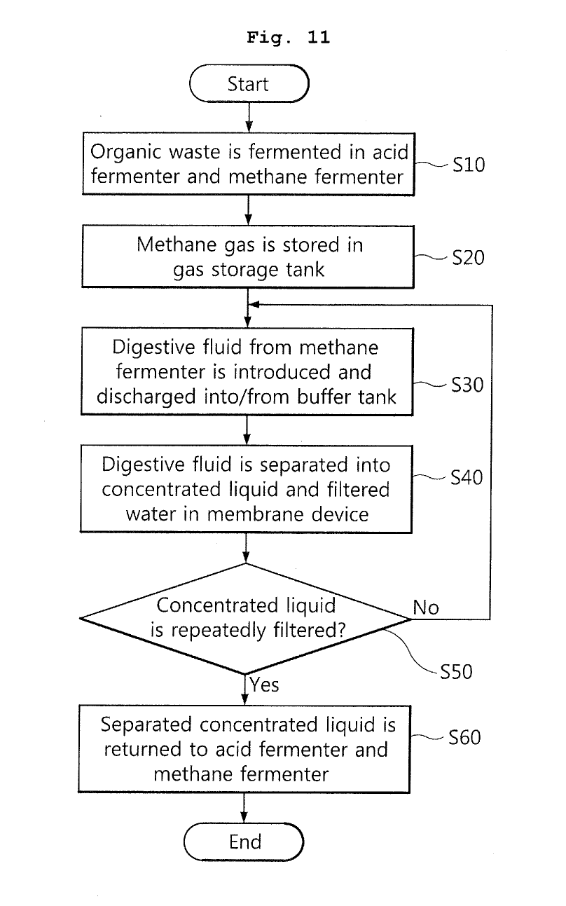

The membrane filtration modules 420 have a cylindrical shape having a predetermined length, in which filtration membranes are mounted. According to the present invention, one or more membrane filtration modules 420 are provided in such a manner as to have both ends protruded outwardly from the frame 410. According to the present invention, as shown in FIG. 1, the membrane filtration modules 420 are made to have four stages, and each stage may be formed of one set of six modules (which is not shown). Accordingly, total 24 membrane filtration modules 420 are needed. Further, as shown in FIG. 10, each membrane filtration module 420 has a plurality of tubular type filtration membranes 424 mounted in a cluster-like shape at the inside of a cylindrical casing 422. The casing 422 has a filtered water discharge hole 423 formed thereon, and the filtered water discharge hole 423 is connected to a filtered water discharge line 411. The symbols “⊚” as shown in FIG. 10 indicate the injection space and direction of the digestive fluid. If the digestive fluid is injected at a given pressure into the tubular type filtration membranes 424, the water contained in the digestive fluid is permeated radially against a cylindrical membrane surface 424a and is thus filtered. Therefore, each of the tubular type filtration membranes 424 is configured to discharge the high concentrated liquid containing digestive microorganisms therein from the opposite side to the injection side and to discharge the filtered water through the filtered water discharge hole 423 formed on the casing 422.

On the other hand, each of the tubular type filtration membranes 424 is formed of ceramic and has a membrane (not shown) coated on the inside of a porous pressure resistant supporting pipe (not shown), the membrane having fine pores formed thereon. Since the tubular type filtration membranes 424 are capable of extending the passage through which the injected raw water flows, they are desirably used in the treatment of the high turbidity raw water. Also, when compared with plate type, hollow fiber type, or spirally wound type, and other types of filtration membranes, advantageously, clogging on the membrane surface 424a occurs less frequently and backwashing is carried out in an easier manner at the time of washing the filtration membranes.

The tubular type filtration membranes 424 make use of ultrafiltration (UF) membranes or microfiltration (MF) membranes.

The injection direction change means serves to change the injection direction of the digestive fluid supplied to the membrane device 400 in a reverse direction to the injection direction at the previously set time. According to the present invention, the injection direction change means includes a second digestive fluid introduction line 402, a second concentrated liquid return line 404, first and second injection valves 401a and 402a, and first and second discharge valves 403a and 404a, and a controller (not shown).

The second digestive fluid introduction line 402 is branched from one side of the first digestive fluid introduction line 401 and is connected to the second header 434 of the membrane device 400. The second concentrated liquid return line 404 connects the first header 432 of the membrane device 400 to one side of the first concentrated liquid return line 403.

The first injection valve 401a is mounted on the first digestive fluid introduction line 401 adjacent to the first header 432, and the first discharge valve 403a is mounted on the first concentrated liquid return line 403 adjacent to the second header 434.

The second injection valve 402a is mounted on the second digestive fluid introduction line 402 adjacent to the second header 434, and the second discharge valve 404a is mounted on the second concentrated liquid return line 404 adjacent to the first header 432.

The first concentrated liquid return line 403 connects the membrane device 400, the acid fermenter 140 and the methane fermenter 150 to each other and serves as a passage through which the concentrated liquid generated in the membrane device 400 is returned to the acid fermenter 140 and the methane fermenter 150. Moreover, the second flow meter 403b is mounted on the first concentrated liquid return line 403 so as to measure the discharge flow rate of the concentrated liquid. In addition thereto, the second manometer 403c is mounted thereon so as to measure the discharge pressure of the concentrated liquid. Further, a valve and a magnetic flow meter may be provided thereon so as to adjust an amount of supply of the concentrated liquid. Also, the first concentrated liquid return line 403 has a circulation valve 403e mounted thereon so as to convey the concentrated liquid discharged from the membrane device 400 to the supply line 301. Under the control of the controller, the circulation valve 403e conducts an open/close operation opposite to the open/close operation carried out by a first supply valve 403f and a second supply valve 403g and also conducts an open/close operation opposite to the open/close operation carried out by a shut-off valve 301a stopping the supply of the methane fermented liquid. That is, if the circulation valve 403e is open to introduce the concentrated liquid conveyed through the first concentrated liquid return line 403 again into the buffer tank 300 and the membrane device 400 through the supply line 301, without entering the acid fermenter 140 and the methane fermenter 150, the first supply valve 403f and the second supply valve 403g become closed. At this time, the shut-off valve 301a becomes closed to stop the digestive fluid discharged from the methane fermenter 150. Contrarily, if the circulation valve 403e is closed to introduce the concentrated liquid conveyed through the first concentrated liquid return line 403 into the acid fermenter 140 and the methane fermenter 150, without entering the supply line 301, the first supply valve 403f and the second supply valve 403g become open. At this time, the shut-off valve 301a becomes open to introduce the digestive fluid discharged from the methane fermenter 150 into the buffer tank 300. In this case, a magnetic flow meter and an auxiliary valve may be further provided on the first concentrated liquid return line 403 so as to adjust an amount of concentrated liquid introduced into the acid fermenter 140 and the methane fermenter 150. Moreover, the magnetic flow meter may be additionally mounted between the methane fermenter 150 and the shut-off valve 301a on the supply line 301 and between the buffer tank 300 and the membrane device 400.

The open/close operations of the first injection valve 401a, the circulation valve 403e, the first supply valve 403f and the second supply valve 403g are controlled by means of the controller. The controller is operated by a program made by the number of filtration times of the concentrated liquid discharged from the membrane device 400. In more detail, the controller is programmed to introduce the low concentrated liquid discharged from the membrane device 400 again into the membrane device 400 through the buffer tank 300 on the supply line 301, without directly entering the acid fermenter 140 and the methane fermenter 150. Also, the first injection valve 401a mounted on the supply line 301 is controlled by the controller so as to introduce a total amount of wash water or low concentrated liquid from the buffer tank 300 into the membrane device 400 through the supply pump 420. In addition to the electrical/electronic equipments like all kinds of pumps and valves mounted on the acid fermenter 140, the methane fermenter 150 and the vortex generating means, the controller controls the open/close operations of the first and second injection valves 401a and 402a and the first and second discharge valves 403a and 404a so as to reversely change the injection direction of the digestive fluid or the low concentrated liquid. At this time, the reverse change period is desirably selected from a range of about one day to three days. That is, a normal injection direction in which the digestive fluid or the low concentrated liquid is conveyed to the first digestive fluid introduction line 401 and the first concentrated liquid return line 403 of the membrane device 400 is periodically changed to a reverse injection direction in which the digestive fluid or the low concentrated liquid is conveyed to the second digestive fluid introduction line 402, the second concentrated liquid return line 404 and the first concentrated liquid return line 403. In this case, the high concentrated liquid means the high concentrated liquid just before supplied to the anaerobic reactor after an initial digestive fluid is repeatedly circulated to the buffer tank 300 and the membrane device 400. Contrarily, the low concentrated liquid means the concentrated liquid being repeatedly circulated to the buffer tank 300 and the membrane device 400 after discharged initially from the membrane device 400. Of course, the digestive fluid is discharged from the methane fermenter 150 and is introduced first into the buffer tank 300.

The discharge tank 500 serves to temporarily store the filtered water discharged from the membrane device 400 and to discharge the filtered water therefrom. As shown in FIG. 3, the discharge tank 500 is connected to the membrane device 400 through the filtered water discharge line 411 and activates a discharge pump 510 to discharge the filtered water through a discharge line 511. On the other hand, the filtered water discharge line 411 has the third flow meters 411a mounted thereon so as to measure a discharge flow rate of the filtered water discharged from the membrane filtration modules 420.

The washing means 600 serves to wash the membrane device 400 so as to extend the life cycle of the membrane device 400. At this time, on the basis of the flow rate values or the pressure values measured through the first and second flow meters 401b and 403b or the first and second manometers 401c and 403c, a flow rate difference or a pressure difference is calculated by the controller, and if the calculated flow rate difference or pressure difference is smaller than a given reference value, the washing means 600 is activated. According to the present invention, as shown in FIG. 1, the washing means 600 includes a wash water supply line 601, a water tank 610, a backwash pump 620, a chemical tank 630 and an air compressor 640, thereby conducting water or chemical washing.

The wash water supply line 601 is connected to the second digestive fluid introduction line 402.

The water tank 610 is connected to the wash water supply line 601 to supply wash water therefrom.

The backwash pump 620 pressurizes and conveys the wash water stored in the water tank 610 to the membrane device 400 through the wash water supply line 601 and the second digestive fluid introduction line 402.

The chemical tank 630 is connected to the wash water supply line 601 to supply washing chemicals therefrom, thereby conducting chemical washing. The washing chemicals make use of NaOCl.

The air compressor 640 is connected to the wash water supply line 601 to supply compressed air therefrom. The air compressor 640 sprays the compressed air to the wash water supply line 601 before the backwash pump 620 is activated, such that the digestive fluid remaining in the membrane device 400 can be discharged. Also, the air compressor 640 generates air bubbles during the water or chemical washing, thereby improving the washing efficiency.

On the other hand, so as to discharge the wash water after the washing of the membrane device 400, first and second wash fluid return lines 405 and 406 and fifth, sixth, and seventh valves 403d, 405a and 406a are further provided.

The fifth valve 403d is mounted on the first concentrated liquid return line 403 and is closed during the washing of the membrane device 400 to allow the wash water to be introduced into the first and second wash fluid return lines 405 and 406.

The first wash fluid return line 405 is branched from the first concentrated liquid return line 403 and has the sixth valve 405a mounted thereon. Further, the first wash fluid return line 405 is branched into two lines 407 and 408 in such a manner as to be connected to a raw water storage tank 412 and the chemical tank 630. At this time, the two lines 407 and 408 have respective valves 407a and 408b mounted thereon. The wash water introduced to the raw water storage tank 412 is supplied to one process of pretreatment.

The second wash fluid return line 406 is branched from the first wash fluid return line 405 and is connected to the discharge tank 500. At this time, the second wash fluid return line 406 has the seventh valve 406a mounted thereon.

Now, an explanation on a method for treating organic waste according to the present invention will be given.

FIG. 11 is a flowchart showing a method for treating organic waste using the apparatus for treating organic waste of FIG. 1.

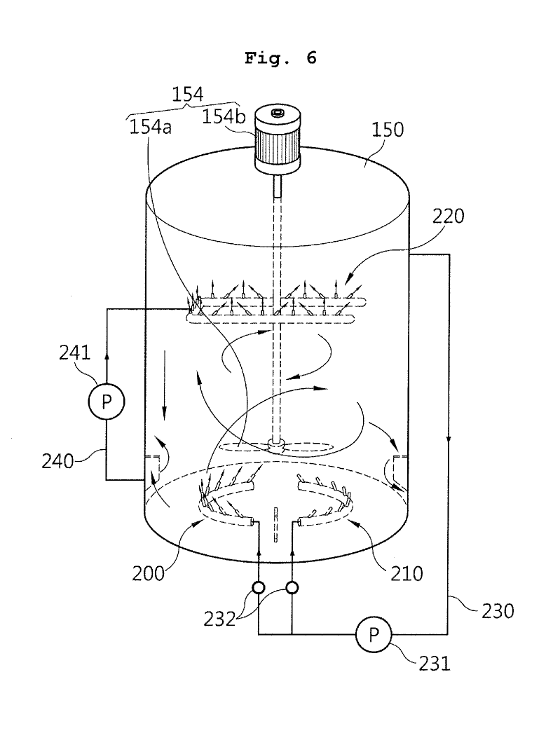

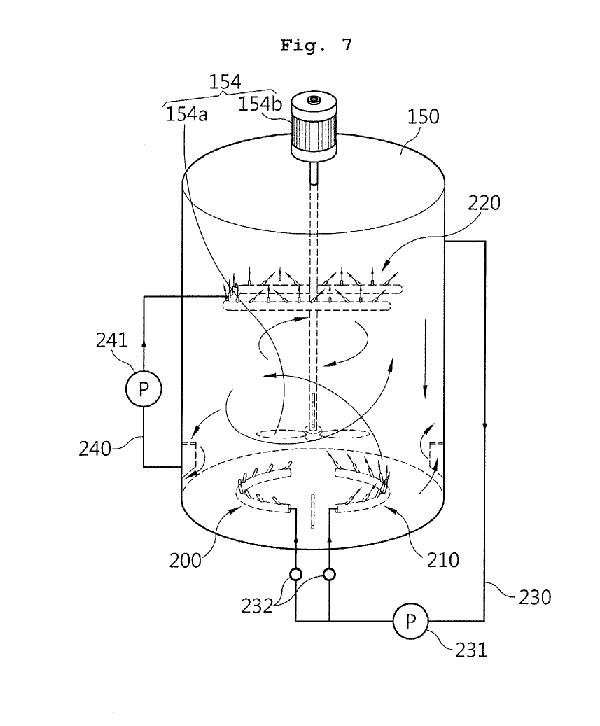

First, the organic waste is supplied to the acid fermenter 140 and the methane fermenter 150 and is then fermented (at step S10). Of course, before the organic waste is introduced into the acid fermenter 140, it is subjected to pretreatment. While the organic waste stays in the acid fermenter 140 for approximately 3 days to 4 days, it is hydrolyzed and acid fermented, such that high molecular organic compound is converted into low molecular organic compound. Also, horizontal water flows are formed inside the acid fermenter 140 by means of the first agitator 142, and the vertical circulation of the horizontal water flow is conducted in the lower portion of the acid fermenter 140 through the agitation circulation line 143, thereby gently achieving the circulation of the upper and lower portions of the acid fermenter 140. In the acid fermenter 140, at this time, the agitation is conducted by means of the first agitator 142 and the vortices formed by means of the agitation circulation line 143. That is, the agitation is carried out in the upper portion of the acid fermenter 140 by means of the first agitator 142 and also the water flows are formed in the lower portion thereof by means of the agitation circulation line 143, such that the oil existing in the upper portion of the acid fermenter 140 is gently agitated with the water flows in the lower portion thereof, thereby accelerating the acid fermentation. Further, as shown in FIGS. 6 and 7, the horizontal water flows are formed inside the methane fermenter 150 by means of the second agitator 154, and the vortices are formed alternatively in the upper and lower portions of the methane fermenter 150 by means of the vortex generating means, such that the vertical and horizontal agitation is conducted in the upper and lower portions of the methane fermenter 150, thereby accelerating the fermentation treatment. At this time, the vortices are generated through the vortex generating means by allowing the engagement valves 232 to open the first nozzle part 200 and the second nozzle part 210 alternatively so as to generate the water flows in the opposite directions to each other. In more detail, in a state where the operation of the second nozzle part 210 stops, the first nozzle part 200 operates to form the water flows in an upward and clockwise direction in the drawings, and then, in a in a state where the operation of the first nozzle part 200 stops, the second nozzle part 210 operates to form the water flows in an upward and counterclockwise direction. Through the vortices generated like this, the horizontal and upward agitation for the anaerobic digestive fluid in the methane fermenter 150 is gently conducted. At this time, the engagement valves 232 are operated under the control of the controller. Additionally, the first vortex circulation line 230 and the second vortex circulation line 240 disposed on the methane fermenter 150 are operated alternatively to generate the vortices alternatively in the upper and lower portions of the methane fermenter 150, thereby naturally conducting the vertical circulation and accelerating the agitation and fermentation. Such alternative operations are also carried out by opening the first and second vortex circulation lines 230 and 240 under the control of the controller. For example, if organic waste of 3 ton/m³ per day is introduced into the methane fermenter 150, the circulation of the organic waste of 3 ton/m³ for one hour is carried out one time by means of the first vortex circulation pump 231, and it is carried out 6 times to 8 times for one day. In the same manner as above, also, the second vortex circulation pump 241 is operated alternatively with the first vortex circulation pump 241, thereby operating the first and second vortex circulation pumps 231 and 241 for 24 hours. As a result, the horizontal agitation and circulatingly vertical agitation are all conducted in the upper and lower portions of the methane fermenter 150. The third nozzle part 220, which is disposed in the upper portion of the methane fermenter 150 and operated in a state of emergency, generates the vortices in the upper portion of the methane fermenter 150 by means of the third nozzles 222. As the third nozzles 222 have vertical or oblique injection angles, vortices are irregularly formed in the upper portion of the methane fermenter 150, thereby enabling the first and second nozzles 202 and 212 to be replaced with the third nozzles 222 in the state of emergency. As a result, the vortices in the upward and clockwise or counterclockwise direction are formed in the lower portion of the methane fermenter 150 by means of the first and second nozzles 202 and 212, and the vortices in the directions different form each other are formed in the upper portion thereof. Further, as the methane fermented organic matters are rotated and lifted by means of the first and second nozzles 202 and 212, the organic matters existing in the upper portion of the methane fermenter 150 are circulated downwardly. In this case, if the third nozzles 222 are disposed on the third nozzle pipe 221 to have the injection angle of 30° to 45° in an upward direction in such a manner as to be operated similarly to the first and second nozzles 202 and 212, the vortices may be generated in the upper portion of the methane fermenter 150. At this time, the third nozzles 222 are operated cooperatively with the first and second nozzles 202 and 212 to permit the vortices to be formed in the lower portion of the methane fermenter 150 in the upward and clockwise direction and in the upper portion thereof in the upward or downward and counterclockwise direction, or reversely. Of course, the vortices are formed differently from those as shown in FIG. 6 or 7. Also, the baffles 260 mounted inside the methane fermenter 150 cause the interferences with the vortices formed by means of the first and second nozzles 202 and 212 and thus form locally irregular vortices, thereby accelerating the agitation in the methane fermenter 150. In addition, the sludge moving along the vortices becomes in contact with the baffles 260 and falls on the bottom surface of the methane fermenter 150, thereby being collected in the sludge grooves 152. Of course, the sludge collected in the sludge grooves 152 is discharged to the outside and removed every given period of time. Moreover, a portion of the organic matters being fermented in the acid fermenter 140 is continuously circulated to a portion of the organic matters being fermented in the methane fermenter 150 along the first and second circulation lines 141 and 151. At this time, the anaerobic digestive fluid of the methane fermenter 150 being circulated to the acid fermenter 140 is supplied by an amount of 10% to 20% of the amount of supply of the acid fermenter 140 to the methane fermenter 150, thereby keeping the organic waste in the acid fermenter 140 in a state of pH 4.3 to pH 4.5 to accelerate the acid fermentation. Further, the decomposition of the organic matters through the anaerobic microorganisms in the acid fermenter 140 is conducted by an amount of 5% to 10% of the amount of acid fermentation decomposition. Of course, the amount of supply of the organic matters to the methane fermenter 150 may be increased to reach a state of pH 5 to pH 6 in accordance with the degrees and environments of the acid fermentation. In this case, the organic matters stay in the acid fermenter 140 for approximately three days to four days and fermented therein, and the organic matters stay in the methane fermenter 140 for approximately 15 days to 20 days to generate the digestive fluid and methane gas.

Next, the methane gas generated from the acid fermenter 140 and the methane fermenter 150 is stored in the gas storage tank 170 (at step S20). The methane gas stored in the gas storage tank 170 is supplied partially to the boiler 180 providing heat to the heaters 160 disposed inside the acid fermenter 140 and the methane fermenter 150, and the methane gas remaining therein is fed to a power system or a high purity gas refinery.

Next, the digestive fluid discharged from the methane fermenter 150 is introduced into the buffer tank 300 (at step S30). The digestive fluid obtained by the reaction of the low molecular organic compound in the methane fermenter 150 is introduced into the buffer tank 300 through the supply line 301 and is agitated therein, thereby preventing the digestive fluid from being settled. The agitation in the buffer tank 300 is carried out by means of the agitator 310 disposed therein to permit the methane gas to be generated therefrom. In this case, when the digestive fluid in the buffer tank 300 is discharged by means of the supply pump 320 from the buffer tank 300, a negative pressure is formed inside the buffer tank 300, and at this time, the gas holder 330 disposed inside the buffer tank 300 becomes expanded to compensate for the negative pressure generated therein. That is, the gas holder 330 becomes expanded by collecting the methane gas generated inside the buffer tank 300, thereby compensating for the positive pressure generated in the buffer tank 300. Of course, if the positive pressure is generated in the buffer tank 300, the gas holder 330 becomes contracted to discharge the methane gas in the gas holder 330 to the outside or to allow a portion of the methane gas therein to stay therein.

After that, the digestive fluid discharged from the buffer tank 300 is conveyed to the membrane device 400 and is separated into the concentrated liquid and the filtered water (at step S40). In more detail, the digestive fluid introduced from the buffer tank 300 is pressurizedly conveyed to the membrane device 400 in a forward direction through the first digestive fluid introduction line 401. At this time, the first injection valve 401a and the first discharge valve 403a are open, and the second injection valve 402a and the second discharge valve 404a are closed. On the other hand, the digestive fluid is distributed through the first header 432 to the membrane filtration modules 420 and is injected thereinto. Further, as shown in FIG. 10, the water contained in the digestive fluid is passed through the tubular type filtration membranes 424 to produce the filtered water, such that the high concentrated liquid containing the digestive microorganisms is discharged from the opposite side to the injection side. The digestive liquid discharged from the membrane filtration modules 420 is collected to the second header 434 and is discharged through the first concentrated liquid return line 403. At the separation step (at the step S40), the injection direction of the digestive fluid is changed every given period of time, thereby extending the life cycle of the membrane filtration modules 420. In more detail, under the control of the controller, the first injection valve 401a and the first discharge valve 403a are closed, and the second injection valve 402a and the second discharge valve 404a are open, thereby reversely changing the injection direction of the digestive fluid. Accordingly, the digestive fluid is introduced into the tubular type filtration membranes 424 through the second digestive fluid introduction line 402 and the second header 434, and the concentrated liquid is discharged to the first concentrated liquid return line 403 through the first header 432 and the second concentrated liquid return line 404. That is, the injection sides of the tubular type filtration membranes 424 are changed, and the pollutants collected on the tubular type filtration membranes 424 are somewhat removed by means of the injection pressure generated in the opposite direction. When compared with the conventional membrane device in which clogging is increasingly caused only on one ends of the filtration membranes, the tubular type filtration membranes 424 according to the present invention have the pollutants collected evenly on the whole membrane surfaces 424a, thereby advantageously delaying the clogging. This permits the life cycle of the tubular type filtration membranes 424 to be extended and reduces the costs for the frequent exchange of the filtration membranes 424. Meanwhile, the period for reversely changing the injection direction of the digestive fluid is desirably selected in a range of approximately one day to three days under the control of the controller.

Next, while the concentrated liquid obtained by the filtration of the membrane device 400 is being discharged through the first concentrated liquid return line 403, it is determined whether the concentrated liquid is in an initially filtered state or in a repeatedly filtered state (at step S50). That is, it is determined whether the concentrated liquid is the low concentrated liquid being in the initially filtered state obtained by the filtration of the methane fermented liquid or the high concentrated liquid being in the repeatedly filtered state obtained by the introduction of the initially filtered concentrated liquid into the membrane device 400 again. At this time, the determination of the filtered state of the concentrated liquid may be based on a given reference concentration of the concentrated liquid, and otherwise, the determination thereof may be based on the number of times of re-filtration of the concentrated liquid discharged from the membrane device 400. If the determination is set on the basis of the given reference concentration of the concentrated liquid, a concentration meter (not shown) is further disposed, and the filtration is repeatedly carried out until the measured concentration reaches the given reference concentration. Otherwise, if the determination is based on the number of times of re-filtration of the concentrated liquid, the number of times of return and filtration of the initially filtered concentrated liquid is based, and generally, the reference of the number of times of re-filtration is one time. Further, the determination may be based on the number of times of open/close operations of the supply valve 320 mounted on the supply line 301 between the buffer tank 300 and the membrane device 400. That is, since the total amount of methane fermented liquid or the concentrated liquid contained in the buffer tank 300 is supplied to the membrane device 400, the number of times of open/close operations of the supply valve 320 is the same as the number of times obtained by adding the number of times of the re-supply of the initially filtered concentrated liquid to the membrane device 400 and open/close operations of the supply valve 320 and the number of times of introduction of the initial methane fermented liquid of one time, and therefore, the determination can be made on the basis of the number of times of open/close operations of the supply valve 320. In this case, if the concentrated liquid is not in the repeatedly filtered state, the methane fermented liquid in the methane fermenter 150 is returned to the introduction step (at the step S30) into the buffer tank 300. At this time, the low concentrated liquid is conveyed from the membrane device 400 to the supply line 301 between the methane fermenter 150 and the buffer tank 300 through the first concentrated liquid return line 403. In this case, the circulation valve 403e mounted on the first concentrated liquid return line 40 is open, and the first supply valve 403f mounted between the first concentrated liquid return line 403 and the acid fermenter 140 and the second supply valve 403g mounted between the first concentrated liquid return line 403 and the methane fermenter 150 are closed. Additionally, the shut-off valve 301a is closed to stop the supply of the digestive fluid from the methane fermenter 150. Of course, the circulation valve 403d is mounted closer to the supply line 301 when compared with the first supply valve 403f and the second supply valve 403g, and the shut-off valve 301a is mounted closer to the methane fermenter 150 when compared with the connected portion of the supply line 301 with the first concentrated liquid return line 403, such that the concentrated liquid can be moved well in accordance with a user's intention.

Next, if the concentrated liquid is in a desired degree of repeatedly filtered state, the concentrated liquid discharged from the second header 434 is returned to the acid fermenter 140 or the methane fermenter 150 through the first concentrated liquid return line 403 (at step S60). As mentioned above, in this case, if the injection direction of the digestive fluid is changed, the concentrated liquid discharged from the second header 434 is returned to the acid fermenter 140 or the methane fermenter 150 through the second concentrated liquid return line 404 and the first concentrated liquid return line 403. At this time, the circulation valve 403e of the first concentrated liquid return line 403 is closed, and the first supply valve 403f and the second supply valve 403g are open, such that the repeatedly filtered high concentrated liquid is introduced into the acid fermenter 140 and the methane fermenter 150. In this case, an amount of concentrated liquid introduced into the acid fermenter 140 and the methane fermenter 150 is adjusted by means of the magnetic meters and auxiliary valves. If necessary, also, the shut-off valve 310a is open to store the methane fermented liquid of the methane fermenter 150 in the buffer tank 300.

On the other hand, the method according to the present invention further includes a washing step for backwashing the tubular type filtration membranes 424 of the membrane device 400. In the separation step (at the step S30), the flow rate of the digestive fluid is measured by means of the first meter 401b mounted on the first digestive fluid introduction line 401 and the flow rate of the concentrated liquid is measured by means of the second meter 403b mounted on the first concentrated liquid return line 403. Based upon the measured values, at this time, a flow rate difference between the introduction flow rate of the digestive fluid and the discharge flow rate of the concentrated liquid is calculated by the controller, and if the flow rate difference value is smaller than the given reference value, the separation step (S40) and the concentrated liquid return step (S60) stop, while the washing step is being conducted to backwash the membrane device 400. In more detail, even though the injection direction of the membrane device 400 is changed, the pollutants become collected on the membrane surfaces 424a of the tubular type filtration membranes 424 as a period of time has been passed, thereby reducing an amount of filtered water and causing the flow rate difference between the introduction flow rate of the digestive fluid and the discharge flow rate of the concentrated liquid to become decreased. Accordingly, when the life cycle of the membrane device 400 is finished or when the performance of the membrane device 400 becomes deteriorated, the backwashing is conducted. At this time, the reference value compared with the flow rate difference calculated by the controller may be set with the data measured in a laboratory, and otherwise, it may be adjusted during real operations. For example, if the digestive fluid of about 5 tons is injected, the filtered water of 0.5 tons and the concentrated liquid of 4.5 tons, as the reference values, are discharged (at a dewatering rate of 100). Under the same principle as above, a pressure difference is calculated between the pressure of the first manometer 401c mounted on the first digestive liquid introduction line 401 and the pressure of the second manometer 403c mounted on the first concentrated liquid return line 403, and if the pressure difference is smaller than the given reference value, the backwashing of the membrane device 400 may be conducted.

The washing step is carried out in the following processes.

First, the air compressor 640 is activated to inject the compressed air into the wash water supply line 601 so as to discharge the digestive fluid remaining therein to the tubular type filtration membranes 424 of the membrane device 400. At this time, the digestive fluid is returned to the acid fermenter 140 and the methane fermenter 150 through the second concentrated liquid return line 404 and the first concentrated liquid return line 403.

After that, the backwash pump 620 is activated to pressurizingly inject the wash water stored in the water tank 610 into the tubular type filtration membranes 424 so as to conduct water washing for a predetermined period of time. At this time, the wash fluid after the washing is introduced into the first concentrated liquid return line 403 through the second concentrated liquid return line 404 and then returned to the first wash fluid return line 405 through the closed fifth valve 403d and to the raw water storage tank 412. At this time, if the wash fluid is satisfied with a given discharge reference, the wash fluid is introduced into the second wash fluid return line 406 and is stored in the discharge tank 500 for discharging. Of course, the wash fluid is conveyed to the first wash fluid introduction line 401, the first concentrated liquid return line 403 and the first wash fluid return line 405, thereby freely changing the movement direction of the wash fluid.

Next, if the washing period in the washing step is smaller than a given reference value, chemicals are added to the wash water for conducting chemical washing. In more detail, even though the water washing for the membrane device 400 is conducted, the washing effects are not perfect, and as a given period of time has been passed, the washing period becomes decreased. For example, if the washing period becomes decreased from a week to three days as the reference value, chemicals are added to the wash water and thus the chemical washing is performed to extend the life cycle of the tubular type filtration membranes 424. However, if the chemical washing is frequently carried out, the fine pores of the tubular type filtration membranes 424 are rapidly damaged and the costs for purchasing the chemicals are increased. Therefore, the chemical washing is not carried out in an initial state of the washing step. The wash fluid after the chemical washing is returned to the chemical tank 630 through the second concentrated liquid return line 404 and the first wash fluid return line 405.

As described above, there is provided the apparatus and method for treating organic waste according to the present invention wherein the organic waste is agitated by the formation of different vortices in the upper and lower portions thereof through the vortex generating means having the plurality of nozzles, thereby optimizing the agitation performance for the organic waste.

Additionally, the nozzles conduct the injection alternatively and the injection directions are opposite to each other, thereby greatly increasing the agitation performance for the organic waste.

Further, the organic waste existing in the upper portions of the acid fermenter and the methane fermenter is circulated to the lower portions thereof, thereby preventing the settlement thereof, and the oil in the organic waste existing in the upper portions thereof is efficiently agitated to improve the contact with the microorganisms.

Furthermore, a portion of the organic matters fermented in the acid fermenter and the methane fermenter is circulated to each other, thereby adjusting the pH, such that the organic matters in the acid fermenter and the methane fermenter are agitated by means of the vortex generating means and the agitators, thereby optimizing the fermentation efficiency.

Additionally, the digestive fluid discharged from the methane fermenter is treated in an indirect circulating manner to be repeatedly circulated to the buffer tank and the membrane device and be thus high concentrated and supplied to the anaerobic reactor, thereby stabilizing the state of the methane fermented liquid in the anaerobic reactor. Further, through the introduction of the high concentrated liquid, the concentrations of the microorganisms and the organic carbon sources fed to the microorganisms are maintained high, thereby accelerating the decomposition of the organic matters and increasing an amount of gas generated.

Moreover, in the state where the movement of the digestive fluid stops, the low concentrated liquid discharged from the membrane device is repeatedly re-circulated to the buffer tank and the membrane device and becomes reduced in volume, thereby improving the agitation capability of the buffer tank. Also, through the reduction of the volume of the low concentrated liquid, the separating efficiency in the membrane device from the concentrated liquid passed through the buffer tank can be improved, thereby producing the high concentrated liquid more improved before.

In addition, the reduction of the concentrations of the microorganisms and the organic carbon sources by the daily circulation amount treated in the anaerobic reactor is suppressed to a maximum degree by means of the high concentrated liquid, thereby improving the ecological environment of the microorganisms.

Further, when the digestive fluid or the concentrated liquid is discharged from the buffer tank, the negative pressure generated therein is compensated by the expansion and contraction of the gas holder, thereby performing the discharging operation gently.

Moreover, the circulation direction of the digestive fluid or the concentrated liquid supplied to the membrane device is changed periodically to decrease a degree of pollution of the tubular type filtration membranes, thereby remarkably increasing the life cycle of the filtration membranes. As a result, the costs caused by the frequent exchange of the high expensive filtration membranes can be reduced and the decrease in the efficiency of the membrane device can be also prevented.

Finally, the membrane device can be efficiently washed through the water washing and the chemical washing, thereby more improving the life cycle of the filtration membranes.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. An apparatus for treating organic waste, in which an acid fermenter, a methane fermenter, a buffer tank and a membrane device are disposed, the apparatus comprising:
a first circulation line having a first circulation pump connected thereon so as to supply a portion of the organic waste being acid fermented in the acid fermenter to the methane fermenter;
a second circulation line having a second circulation pump connected thereon so as to supply a portion of anaerobic digestive fluid methane fermented in the methane fermenter to the acid fermenter; and
vortex generating means having a plurality of first, second and third nozzles disposed in the methane fermenter so as to allow the anaerobic digestive fluid in the methane fermenter to be agitated by vortices generated thereof,
wherein the vortex generating means comprises:
a first nozzle part having the plurality of first nozzles disposed at a given angle on a first nozzle pipe so as to form water flows in one direction in the anaerobic digestive fluid fermented in the methane fermenter;
a second nozzle part having the plurality of second nozzles disposed at a given angle on a second nozzle pipe so as to form water flows in the opposite direction to the injection direction of the first nozzle part; and
a first vortex circulation line adapted to supply the anaerobic digestive fluid on the upper portion of the methane fermenter to the first and second nozzle pipes,
whereby the water flows are formed alternatively in the one direction and opposite direction to the one direction to generate vortices in the methane fermenter.

2. The apparatus for treating organic waste according to claim 1, further comprising:
a sorting crusher adapted to sort and crush the organic waste;
a mill adapted to grind the crushed organic waste through the sorting crusher to appropriate sizes capable of being introduced into the acid fermenter;
a screen adapted to filter the organic waste of more than a predetermined size from the organic waste passed through the mill;
a storage tank adapted to temporarily store the organic waste passed through the screen;
heaters disposed in the acid fermenter and the methane fermenter so as to raise the organic waste in the acid fermenter and the methane fermenter up to a given temperature;
a gas storage tank adapted to store the methane gas generated from the methane fermenter therein; and
a boiler adapted to receive the methane gas stored in the gas storage tank so as to supply heat to the heaters.

3. The apparatus for treating organic waste according to claim 1, wherein the first vortex circulation line comprises:
a first vortex circulation pump connected thereon to supply the anaerobic digestive fluid on the upper portion of the methane fermenter to the first and second nozzle pipes; and
a pair of engagement valves adapted to open the first and second nozzle pipes alternatively.

4. The apparatus for treating organic waste according to claim 1, wherein the first and second nozzle pipes are disposed on the bottom portion of the methane fermenter in such a manner as to be bent at a given curvature toward the center of the methane fermenter.

5. The apparatus for treating organic waste according to claim 1, wherein the plurality of first nozzles and second nozzles has an upward injection angle in a range of 30° to 45°.

6. The apparatus for treating organic waste according to claim 1, wherein the vortex generating means further comprises:
a third nozzle part having the plurality of third nozzles disposed at a given angle on a third nozzle pipe located on the upper portion of the methane fermenter; and
a second vortex circulation line adapted to supply the anaerobic digestive fluid on the lower portion of the methane fermenter to the third nozzle pipe.

7. The apparatus for treating organic waste according to claim 6, wherein the second vortex circulation line has a second vortex circulation pump connected thereon so as to supply the anaerobic digestive fluid on the lower portion of the methane fermenter to the third nozzle pipe.

8. The apparatus for treating organic waste according to claim 6, wherein the plurality of third nozzles have a given upward and vertical or oblique injection angle.

9. The apparatus for treating organic waste according to claim 6, wherein the plurality of third nozzles have an upward or downward injection angle in a range of 30° to 45°.

10. The apparatus for treating organic waste according to claim 7, wherein the vortex generating means further comprises a controller adapted to control the first vortex circulation pump and the second vortex circulation pump in such a manner as to be operated alternatively and to control the engagement valves in such a manner as to operate the first nozzle part and the second nozzle part alternatively.

11. The apparatus for treating organic waste according to claim 1, wherein the vortex generating means further comprises a plurality of baffles disposed along the side surface of the lower portion of the methane fermenter so as to provide interferences to the flows of the vortices.

12. The apparatus for treating organic waste according to claim 11, wherein the methane fermenter comprises:
a plurality of sludge grooves formed along the edge portion of the bottom surface thereof so as to collect the sludge falling down by the contact with the baffles; and
sludge discharging lines connected to the sludge grooves so as to discharge sludge therethrough.

13. The apparatus for treating organic waste according to claim 1, wherein the bottom surface of the methane fermenter is inclined downwardly from the center to the edge side thereof.

14. The apparatus for treating organic waste according to claim 1, wherein the methane fermenter further comprises a second agitator having second agitating wings disposed therein and a second agitating motor for rotating the second agitating wings.

15. The apparatus for treating organic waste according to claim 1, wherein the acid fermenter comprises a first agitator having first agitating wings disposed therein and a first agitating motor for rotating the first agitating wings.

16. The apparatus for treating organic waste according to claim 1, wherein the acid fermenter further comprises an agitation circulation line adapted to connect the upper and lower portions thereof and having an agitation circulation pump connected thereon so as to supply the organic waste from the upper portion thereof to the lower portion thereof.

17. The apparatus for treating organic waste according to claim 1 of 3, wherein the first circulation line is connected to the first vortex circulation line from the acid fermenter.

18. The apparatus for treating organic waste according to claim 17, wherein the connected point between the first circulation line and the first vortex circulation line is disposed before the engagement valves.

19. The apparatus for treating organic waste according to claim 16, wherein the second circulation line is connected to the agitation circulation line from the methane fermenter.

20. The apparatus for treating organic waste according to claim 1, wherein the buffer tank comprises a gas holder disposed on the upper portion thereof so as to compensate for a negative pressure generated when the digestive fluid stored therein is discharged.

21. The apparatus for treating organic waste according to claim 20, wherein the gas holder is formed of a soft material, and if the negative pressure is generated inside the buffer tank, the gas holder is contracted through the discharging of the gas collected therein.

22. The apparatus for treating organic waste according to claim 1, further comprising injection direction change means adapted to reversely change the injection direction of the digestive fluid supplied to the membrane device every given period of time, the injection direction change means comprising:
a second digestive fluid introduction line branched from a first digestive fluid introduction line connected to the buffer tank and connected to the other end of the membrane device;
a second concentrated liquid return line connected to the membrane device so as to connect a first concentrated liquid return line disposed to supply concentrated liquid to the acid fermenter and the methane fermenter thereto;
a first injection valve mounted on the first digestive fluid introduction line; a first discharge valve mounted on the first concentrated liquid return line;
a second injection valve mounted on the second digestive fluid introduction line; a second discharge valve mounted on the second concentrated liquid return line; and
a controller adapted to control a supply pump mounted on the first digestive fluid introduction line, the first and second injection valves, and the first and second discharge valves.

23. The apparatus for treating organic waste according to claim 22, wherein the first concentrated liquid return line is connected to a supply line so as to circulate the concentrated liquid to the buffer tank.

24. The apparatus for treating organic waste according to claim 23, further comprising:
a circulation valve mounted on the first concentrated liquid return line so as to introduce the concentrated liquid conveyed through the first concentrated liquid return line into the supply line or to stop the introduction into the supply line;
a first supply valve mounted on the first concentrated liquid return line so as to introduce the concentrated liquid conveyed through the first concentrated liquid return line into the acid fermenter or to stop the introduction into the acid fermenter;
a second supply valve mounted on the first concentrated liquid return line so as to introduce the concentrated liquid conveyed through the first concentrated liquid return line into the methane fermenter or to stop the introduction into the methane fermenter; and
a shut-off valve adapted to be open and closed so as to stop the movement of the digestive fluid discharged from the methane fermenter.

25. The apparatus for treating organic waste according to claim 23, wherein if the circulation valve is open, the shut-off valve, the first supply valve and the second supply valve become closed, and if the circulation valve is closed, the first supply valve and the second supply valve become open.

26. The apparatus for treating organic waste according to claim 25, wherein the shut-off valve is open only when the digestive fluid is supplied to the buffer tank.

27. The apparatus for treating organic waste according to claim 22, wherein the membrane device comprises:
a frame;
one or more membrane filtration modules disposed on the frame and each having a casing having a discharge hole formed to discharge filtered water therethrough and a plurality of tubular type filtration membranes mounted inside the casing;
a first header disposed on one ends of the membrane filtration modules and connected to the first digestive fluid introduction line; and
a second header disposed on the other ends of the membrane filtration modules and connected to the first concentrated liquid return line.

28. The apparatus for treating organic waste according to claim 27, wherein the tubular type filtration membranes make use of ultrafiltration (UF) membranes or microfiltration (MF) membranes.

29. The apparatus for treating organic waste according to claim 22, wherein the membrane device further comprises:
a first flow meter mounted on the first digestive fluid introduction line so as to measure an injection flow rate of the digestive fluid; and
a second flow meter mounted on the first concentrated liquid return line so as to measure a discharge flow rate of the concentrated liquid.

30. The apparatus for treating organic waste according to claim 22, wherein the membrane device further comprises:
a first manometer mounted on the first digestive fluid introduction line so as to measure an injection pressure of the digestive fluid; and
a second manometer mounted on the first concentrated liquid return line so as to measure a discharge pressure of the digestive fluid.

31. The apparatus for treating organic waste according to claim 29, wherein the membrane device further comprises third flow meters adapted to measure a discharge flow rate of the filtered water discharged from the membrane filtration modules.

32. The apparatus for treating organic waste according to claim 29, wherein the membrane device further comprises washing means adapted to backwash the membrane device in such a manner as to be activated when a flow rate difference between the flow rate values measured through the first and second flow meters is calculated by the controller and is smaller than a given reference value.

33. The apparatus for treating organic waste according to claim 32, wherein the washing means comprises:
a wash water supply line connected to the second digestive fluid introduction line;
a water tank connected to the wash water supply line; and
a backwash pump adapted to pressurize and convey the wash water stored in the water tank to the wash water supply line.

34. The apparatus for treating organic waste according to claim 33, wherein the washing means further comprises at least one of:
a discharge tank adapted to temporarily store the filtered water discharged from the membrane device;
a chemical tank adapted to add washing chemicals to the wash water supply line; and
an air compressor adapted to supply compressed air to the wash water supply line.

35. The apparatus for treating organic waste according to claim 34, wherein the washing means further comprises:
a fifth valve mounted on the first concentrated liquid return line in such a manner as to be closed during the backwashing; and
a first wash fluid return line branched from the first concentrated liquid return line so as to introduce the wash water thereinto when the fifth valve is closed in such a manner as to be connected to a raw water storage tank and the chemical tank.

36. A method for treating organic waste comprising:
a first step wherein the organic waste is supplied and fermented to an acid fermenter and a methane fermenter;
a second step wherein the methane gas generated in the first step is stored in a gas storage tank;
a third step wherein the digestive fluid discharged from the methane fermenter or the concentrated liquid discharged from a membrane device is introduced and discharged into/from a buffer tank through a supply line;
a fourth step wherein the digestive fluid stored in the buffer tank is injected into the membrane device by means of a supply pump and is separated into the concentrated liquid and filtered water and wherein the injection direction of the digestive fluid is changed reversely every given period of time; and
a sixth step wherein the separated concentrated liquid is returned to the acid fermenter and the methane fermenter.

37. The method for treating organic waste according to claim 36, wherein in the first step vortices are formed in a clockwise or counterclockwise direction by means of a plurality of first and second nozzles, and vortices are formed in different directions by means of a plurality of third nozzles.

38. The method for treating organic waste according to claim 36, wherein in the first step a portion of the digestive fluid of the methane fermenter is circulated to the acid fermenter through a second circulation line connecting the acid fermenter and the methane fermenter, thereby being maintained in a range of pH4.0 to pH4.5.

39. The method for treating organic waste according to claim 36, wherein in the third step the negative pressure generated when the digestive fluid is discharged from the buffer tank is compensated by the contraction of a gas holder through the gas discharging therefrom, the gas holder being disposed inside the buffer tank in such a manner as to be expanded by collecting the gas generated in the buffer tank.

40. The method for treating organic waste according to claim 36, wherein the fourth step further comprises the steps of:
measuring a flow rate difference or a pressure difference between both ends of tubular type filtration membranes disposed in the membrane device;
comparing the measured difference with a given reference value;
after the comparing result, if the flow rate different or the pressure difference is smaller than the given reference value, stopping the fourth and sixth steps; and
backwashing the tubular type filtration membranes for a given period of time.

41. The method for treating organic waste according to claim 40, wherein the backwashing step comprises:
a water washing step of pressurizing and injecting wash water stored in a water tank into the tubular type filtration membranes by means of the activation of a backwash pump so as to perform water washing; and
a chemical washing step of adding chemicals to the wash water so as to perform chemical washing if a washing period in the water washing step is smaller than a given reference value.

42. The method for treating organic waste according to claim 41, wherein the backwashing step further comprises the steps of:
returning the wash water after the water washing to a raw water storage tank; and
returning the wash water after the chemical washing to a chemical tank.

43. The method for treating organic waste according to claim 36, further comprising a fifth step wherein it is determined whether the concentrated liquid discharged from the membrane device is repeatedly filtered.

44. The method for treating organic waste according to claim 43, wherein in the fifth step if the concentrated liquid is not repeatedly filtered, it is returned to the third step wherein the methane fermented liquid is introduced into the buffer tank through the supply line.

45. The method for treating organic waste according to claim 43, wherein in the fifth step if the concentrated liquid is repeatedly filtered, it is moved to the sixth step.
